(19) United States
(12) Patent Application Publication
(10) Pub. No.: US 2014/0288156 A1
(43) Pub. Date: Sep. 25, 2014

(54) METHODS FOR THE TREATMENT AND DIAGNOSIS OF ATHEROSCLEROSIS

(71) Applicants: INSERM (INSTITUT NATIONAL DE LA SANTE ET DE LA RECHERCHE MEDICALE), Paris (FR); UNIVERSITY PARIS DESCARTES (PARIS V), Paris (FR); UNIVERSITE PARIS SUD - PARIS XI, Orsay Cedex (FR)

(72) Inventors: Alain Tedgui, Paris (FR); Xavier Loyer, Paris (FR); Jean MacCario, Villejuif Cedex (FR)

(21) Appl. No.: 14/353,305

(22) PCT Filed: Oct. 29, 2012
(86) PCT No.: PCT/EP2012/071411

§ 371 Date: Apr. 22, 2014

(30) Foreign Application Priority Data
Oct. 27, 2011 (EP) 11306396.0

(51) Int. Cl. C12N 15/113; C12Q 1/68

(52) U.S. Cl. CPC C12N 15/113; C12Q 1/6883
USPC 514/44 A; 514/44 R; 435/6.12; 435/6.11; 506/9

(57) ABSTRACT

The present invention relates to the treatment and the diagnosis of atherosclerosis, in particular to a miRNA for use in the treatment and the diagnosis of atherosclerosis.

### FIELD OF THE INVENTION

The present invention relates to the treatment and diagnosis of atherosclerosis.

### BACKGROUND OF THE INVENTION

Atherosclerosis is the most common cause of death in western societies and is predicted to become the leading cause of cardiovascular disease in the world within two decades. Atherosclerosis contributes to the development of atherosclerotic vascular diseases (AVD) which may affect the coronary arteries (causing ischaemic heart disease), the cerebral circulation (causing cerebrovascular disease), the aorta (producing aneurysms that are prone to thrombosis and rupture) and peripheral blood vessels, typically the legs (causing peripheral vascular disease and intermittent claudication). Ischaemic heart disease (IHD) includes angina (chest pain caused by insufficient blood supply to cardiac muscle) and myocardial infarction (death of cardiac muscle) and cerebrovascular disease includes stroke and transient ischaemic attacks. One in three men and one in four women will die from IHD and the death rate for IHD was 58 per 100,000 in 1990.

So, there is a recognized and permanent need in the art for new reliable methods for treating atherosclerosis.

The different steps of atherogenesis comprise activation and dysfunction of endothelial cells, adhesion, migration and activation of leukocytes in the vascular wall, subendothelial lipoprotein retention and modification into proatherogenic particles, transformation of monocytes into macrophage foam cells and deposition of atheromatous lipids. Following the accumulation of additional inflammatory cell subsets and extracellular lipids, the early atherosclerotic plaques then progress into mature plaques. These latter can become necrotic, fibrous, ultimately resulting in plaque rupture, and leading to arterial occlusion and myocardial infarction or stroke.

Hemodynamics, specifically, fluid shear stress, modulates the focal nature of atherosclerosis. Shear stress induces vascular oxidative stress via the activation of membrane-bound NADPH oxidases present in vascular smooth muscle cells, fibroblasts, and phagocytic mononuclear cells. Shear stress acting on the endothelial cells at arterial bifurcations or branching points regulates both NADPH oxidase and nitric oxide (NO) synthase activities. The circulating oxidized low-density lipoprotein (ox-LDL) particles play an important role in assessing vascular oxidative stress.

In this way, it has been suggested that characterisation of new therapeutic targets that are modulated by shear stress and oxLDL stimulation may be highly desirable.

### SUMMARY OF THE INVENTION

The present invention relates to the treatment and diagnosis of atherosclerosis.

### DETAILED DESCRIPTION OF THE INVENTION

Endothelial miRNAs expression profile was investigated by inventors using microarray analysis under different flow conditions and oxLDL stimulation. Human endothelial cells (HUVEC) were exposed to low shear stress (LSS) and high shear stress (HSS). To mimic pro-atherogenic conditions in vitro, inventors also performed stimulation with oxidized LDL in both conditions (LSS and HSS). All conditions were compared to static conditions treated or not with oxLDL. The inventors surprisingly found that an amount of miRNAs is modulated by flow conditions (shear stress) and by oxLDL stimulation.

### Therapeutic Methods

The inventors demonstrated that the miRNAs of Table A can be divided in two groups. Indeed, under pro-atherogenic conditions, flow conditions (shear stress) and oxLDL stimulation, 3 miRNAs are overexpressed (i.e. miR-21, miR320c and miR-1908) whereas 3 miRNAs are down expressed (i.e. miR-302c, miR-372 and miR-624). Accordingly, the modulation (i.e. activation or inhibition) of the expression of said miRNAs represent a suitable method for the treatment of an atherosclerosis in a patient. For example, if the expression level of miRNAs in patient having or at risk of having or developing an atherosclerosis are downregulated, then the atherosclerosis can be treated by raising the expression level of said miRNAs. Likewise, if the expression level of miRNAs associated with an atherosclerosis is up-regulated in a patient, then the atherosclerosis can be treated by reducing the expression level of said miRNAs.

Accordingly, the present invention relates to a method for the treatment of atherosclerosis in a patient in need thereof comprising administering said patient with a therapeutically effective amount of i) a compound that raises the expression level of one miRNA selected from the group consisting of miR-146a, miR-302c, miR-372 and miR-624 or ii) a compound that inhibits the expression level of one miRNA selected from the group consisting of miR-92b, miR-126, miR-181a, miR-320c-1, miR-320c-2 and miR-1908. Combination of compounds that raise the expression level of said miRNAs and compounds that inhibit the expression level of said miRNAs are also encompassed by the invention for use in the treatment of atherosclerosis in a patient in need thereof.

In one embodiment, the method of the invention may further comprise administering said patient with a therapeutically effective amount of a compound that inhibits the expression level of one miRNA selected from the group consisting of miR-21, miR-92a-1, miR-92a-2. Combination of compounds that inhibit the expression level of said miRNAs are also encompassed by the invention for use in the treatment of atherosclerosis in a patient in need thereof.

The term “miRNAs” has its general meaning in the art and refers to microRNA molecules that are generally 21 to 22 nucleotides in length, even though lengths of 19 and up to 23 nucleotides have been reported. miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease Ill-like nuclease enzyme called Dicer. The processed miRNA is typically a portion of the stem. The processed miRNA (also referred to as “mature miRNA”) become part of a large complex to down-regulate a particular target gene. All the miRNAs pertaining to the invention are known per se and sequences of them are publicly available from the data base http://microrna.sanger.ac.uk/sequences/. The miRNAs of the invention are listed in Table A:

TABLE A
list of the miRNAs according to the invention

| miRNA | miRBase Accession number |
|---|---|
| miRNA-21 | MI0000077 |
| miRNA-92a-1 | MI0000093 |
| miRNA-92a-2 | MI0000094 |
| miRNA-92b | MI0000379 |
| miRNA-126 | MI0000471 |
| miRNA-146a | MI0000477 |
| miRNA-155 | MI0000481 |
| miRNA-181a | MI0000289 |
| miRNA-302c | MI0000773 |
| miRNA-320c-1 | MI0003778 |
| miRNA-320c-2 | MI0008191 |
| miRNA-372 | MI0000780 |
| miRNA-624 | MI0003638 |
| miRNA-1908 | MI0008329 |

As used herein, the term “patient” denotes a mammal. In a preferred embodiment of the invention, a patient according to the invention refers to any patient (preferably human) afflicted with or susceptible to be afflicted with atherosclerosis.

In one embodiment of the invention, a patient refers to any patient afflicted with coronary disorder, vascular disorders, atherosclerotic vascular disease, such as aneurysm or stroke, asymptomatic coronary artery diseases, chronic ischemic disorders without myocardial necrosis, such as stable or effort angina pectoris; acute ischemic disorders myocardial necrosis, such as unstable angina pectoris; and ischemic disorders such as myocardial infarction.

As used herein, the term “sample” refers to any tissue sample derived from the patient that contains nucleic acid materials. Said tissue sample is obtained for the purpose of the in vitro evaluation. The sample can be fresh, frozen, fixed (e.g., formalin fixed), or embedded (e.g., paraffin embedded). In a particular embodiment the sample results from biopsy performed in the tissue sample of the patient. For example an endothelial biopsy performed in the patient affected by an atherosclerosis. In a particular embodiment the sample can be blood, serum, urine or saliva.

Accordingly the present invention also relates to a compound that raises the expression level of one miRNA selected from the group consisting of miR-146a, miR-302c, miR-372 and miR-624 for use in the treatment of atherosclerosis in a patient in need thereof. Combination of compounds that raise the expression level of said miRNAs are also encompassed by the invention.

In one embodiment, the present invention also relates to a compound that inhibits the expression level of one miRNA selected from the group consisting of miR-92b, miR-126, miR-181a, miR-320c-1, miR-320c-2 and miR-1908 for use in the treatment of atherosclerosis in a patient in need thereof. Combination of compounds that inhibit the expression level of said miRNAs are also encompassed by the invention for use in the treatment of atherosclerosis.

In one embodiment, the present invention also relates to a compound that inhibits the expression level of one miRNA selected from the group consisting of miR-21, miR-92a-1, miR-92a-2 for use in the treatment of atherosclerosis in a patient in need thereof. Combination of compounds that inhibit the expression level of said miRNAs are also encompassed by the invention for use in the treatment of atherosclerosis.

In one embodiment, the present invention relates to a compound that raises the expression level of one miRNA selected from the group consisting of miR-146a, miR-302c, miR-372, miR-624 or a compound that inhibits the expression level of one miRNA selected from the group consisting of miR-92b, miR-126, miR-181a, miR-320c-1, miR-320c-2 and miR-1908 or a combination thereof for use in the treatment of atherosclerosis in a patient in need thereof.

In one embodiment, the present invention also relates to a compound according to the invention in combination with a compound that inhibits the expression level of one miRNA selected from the group consisting of miR-21, miR-92a-1, miR-92a-2 or a combination thereof for use in the treatment of atherosclerosis in a patient in need thereof.

As used herein, a “therapeutically effective amount” of a compound of the invention is an amount sufficient to prevent or to treat atherosclerosis in a patient at a reasonable benefit/risk ratio applicable to any medical treatment.

One skilled in the art can readily determine an effective amount of said compound to be administered to a given patient, by taking into account factors such as the size and weight of the patient; the extent of disease penetration; the age, health and sex of the patient; the route of administration; and whether the administration is regional or systemic. An effective amount of said compound can be based on the approximate or estimated body weight of a patient to be treated. Preferably, such effective amounts are administered parenterally or enterally, as described herein. For example, an effective amount of the compound is administered to a patient can range from about 5-3000 micrograms/kg of body weight, and is preferably between about 700-1000 micrograms/kg of body weight, and is more preferably greater than about 1000 micrograms/kg of body weight. One skilled in the art can also readily determine an appropriate dosage regimen for the administration of the compound to a given patient. For example, the compound can be administered to the patient once (e.g., as a single injection or deposition). Alternatively, said compound can be administered once or twice daily to a patient for a period of about three to twenty-eight days, more preferably about seven to ten days. In a preferred dosage regimen, the compound is administered once a day for seven days. Where a dosage regimen comprises multiple administrations, it is understood that the effective amount of the compound administered to the patient can comprise the total amount of compounds administered over the entire dosage regimen.

In a particular embodiment, the compound that raises the expression level of one miRNA selected from the group consisting of miR-146a, miR-302c, miR-372 and miR-624 may consist in an isolated miRNA selected from the group consisting of isolated miR-146a, miR-302c, miR-372 and miR-624.

As used herein, an “isolated” miRNA is one which is synthesized, or altered or removed from the natural state through human intervention. For example, a miRNA naturally present in a living animal is not “isolated.” A synthetic miRNA, or a miRNA partially or completely separated from the coexisting materials of its natural state, is “isolated.” An isolated miRNA can exist in substantially purified form, or can exist in a cell into which the miRNA has been delivered. Thus, a miRNA which is deliberately delivered to, or expressed in, a cell is considered an “isolated” miRNA. A miRNA produced inside a cell by from a miRNA precursor molecule is also considered to be “isolated” molecule.

Isolated miRNAs can be obtained using a number of standard techniques. For example, the miRNAs can be chemically synthesized or recombinantly produced using methods known in the art. Preferably, miRNAs are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., USA), Pierce Chemical (part of Perbio Science, Rockford, 111., USA), Glen Research (Sterling, Va., USA), ChemGenes (Ashland, Mass., USA) and Cruachem (Glasgow, UK).

In some embodiments, of the invention, a synthetic miRNA contains one or more design elements. These design elements include, but are not limited to: (i) a replacement group for the phosphate or hydroxyl of the nucleotide at the 5′ terminus of the complementary region; (ii) one or more sugar modifications. In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′oxygen-methyl), DMTO (4,4′-dimethoxytrityl with oxygen), fluorescein, a thiol, or acridine, though other replacement groups are well known to those of skill in the art and can be used as well. In particular embodiments, the sugar modification is a 2′O-Me modification. In further embodiments, there is one or more sugar modifications in the first or last 2 to 4 residues of the complementary region or the first or last 4 to 6 residues of the complementary region.

In a particular embodiment, the compounds that raises the expression level of miRNAs of the invention is resistant to degradation by nucleases. One skilled in the art can readily synthesize nucleic acids which are nuclease resistant, for example by incorporating one or more ribonucleotides that are modified at the 2′-position into the miRNAs. Suitable 2′-modified ribonucleotides include those modified at the 2′-position with fluoro, amino, alkyl, alkoxy, and O-allyl.

The present invention also relates to a vector comprising a nucleic acid according to the invention for use in the treatment of atherosclerosis in a patient in need thereof.

Alternatively, the miRNAs can be expressed from recombinant circular or linear DNA plasmids using any suitable promoter. Suitable promoters for expressing RNA from a plasmid include, e.g., the U6 or HI RNA pol III promoter sequences, or the cytomegalovirus promoters. Selection of other suitable promoters is within the skill in the art. The recombinant plasmids of the invention can also comprise inducible or regulatable promoters for expression of the miRNAs in cardiovascular cells.

The miRNAs that are expressed from recombinant plasmids can be isolated from cultured cell expression systems by standard techniques. The miRNAs which are expressed from recombinant plasmids can also be delivered to, and expressed directly in, the cardiovascular cells. The use of recombinant plasmids to deliver the miRNAs to cardiovascular cells is discussed in more detail below.

The miRNAs can be expressed from a separate recombinant plasmid, or can be expressed from a unique recombinant plasmid. Preferably, the miRNAs are expressed as the RNA precursor molecules from a single plasmid, and the precursor molecules are processed into the functional miRNA by a suitable processing system, including processing systems extant within a cardiovascular cell. Other suitable processing systems include, e.g., the in vitro Drosophila cell lysate system as described in U.S. published application 2002/0086356 to Tuschl et al. and the E. coli RNAse III system described in U.S. published patent application 2004/0014113 to Yang et al., the entire disclosures of which are herein incorporated by reference.

Selection of plasmids suitable for expressing the miRNAs, methods for inserting nucleic acid sequences into the plasmid to express the gene products, and methods of delivering the recombinant plasmid to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee et al. (2002), Nat. Biotechnol. 20:500-505; and Paul et al. (2002), Nat. Biotechnol. 20:505-508, the entire disclosures of which are herein incorporated by reference.

In one embodiment, a plasmid expressing the miRNAs comprises a sequence encoding a miR precursor RNA under the control of the CMV intermediate early promoter. As used herein, “under the control” of a promoter means that the nucleic acid sequences encoding the miRNA are located 3′ of the promoter, so that the promoter can initiate transcription of the miRNA coding sequences.

The miRNAs can also be expressed from recombinant viral vectors. It is contemplated that the miRNAs can be expressed from separate recombinant viral vectors, or from a unique viral vector. The RNA expressed from the recombinant viral vectors can either be isolated from cultured cell expression systems by standard techniques, or can be expressed directly in cardiovascular cells. The use of recombinant viral vectors to deliver the miRNAs to cardiovascular cells is discussed in more detail below.

The recombinant viral vectors of the invention comprise sequences encoding the miRNAs and any suitable promoter for expressing the miRNAs sequences. Suitable promoters include, for example, the U6 or HI RNA pol III promoter sequences, or the cytomegalovirus promoters. Selection of other suitable promoters is within the skill in the art. The recombinant viral vectors of the invention can also comprise inducible or regulatable promoters for expression of the miRNAs in cardiovascular cells.

Any viral vector capable of accepting the coding sequences for the miRNAs can be used; for example, vectors derived from adenovirus (AV); adenoassociated virus (AAV); retroviruses (e.g., lentiviruses (LV), Rhabdoviruses, murine leukemia virus); herpes virus, and the like. The tropism of the viral vectors can be modified by pseudotyping the vectors with envelope proteins or other surface antigens from other viruses, or by substituting different viral capsid proteins, as appropriate. For example, lentiviral vectors of the invention can be pseudotyped with surface proteins from vesicular stomatitis virus (VSV), rabies, Ebola, Mokola, and the like. AAV vectors of the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes are within the skill in the art; see, e.g., Rabinowitz J. E. et al. (2002), J Virol 76:791801, the entire disclosure of which is herein incorporated by reference.

Selection of recombinant viral vectors suitable for use in the invention, methods for inserting nucleic acid sequences for expressing RNA into the vector, methods of delivering the viral vector to the cells of interest, and recovery of the expressed RNA products are within the skill in the art. See, for example, Dornburg (1995), Gene Therap. 2:301-310; Eglitis (1988), Biotechniques 6:608-614; Miller (1990), Hum. Gene Therap. 1:5-14; and Anderson (1998), Nature 392:25-30, the entire disclosures of which are herein incorporated by reference.

Preferred viral vectors are those derived from AV and AAV. A suitable AV vector for expressing the miRNAs, a method for constructing the recombinant AV vector, and a method for delivering the vector into target cells, are described in Xia et al. (2002), Nat. Biotech. 20:1006-1010, the entire disclosure of which is herein incorporated by reference. Suitable AAV vectors for expressing the miRNAs, methods for constructing the recombinant AAV vector, and methods for delivering the vectors into target cells are described in Samulski et al. (1987), J. Virol. 61:3096-3101; Fisher et al. (1996), J. Virol., 70:520-532; Samulski et al. (1989), J. Virol. 63:3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference. Preferably, the miRNAs are expressed from a single recombinant AAV vector comprising the CMV intermediate early promoter.

In one embodiment, a recombinant AAV viral vector of the invention comprises a nucleic acid sequence encoding a miR precursor in operable connection with a polyT termination sequence under the control of a human U6 RNA promoter. As used herein, “in operable connection with a polyT termination sequence” means that the nucleic acid sequences encoding the sense or antisense strands are immediately adjacent to the polyT termination signal in the 5′ direction. During transcription of the miRNA sequences from the vector, the polyT termination signals act to terminate transcription.

In the practice of the present treatment methods, an effective amount of at least one compound which inhibits miRNA expression can also be administered to the patient. As used herein, “inhibiting miRNA expression” means that the production of miRNA from the miRNA in the endothelial cell after treatment is less than the amount produced prior to treatment. One skilled in the art can readily determine whether miRNA expression has been inhibited in a endothelial cell, using for example the techniques for determining miRNA transcript level discussed above for the diagnostic methods.

Suitable compounds for inhibiting miRNA expression include double-stranded RNA (such as short- or small-interfering RNA or “siRNA”), antagomirs, antisense nucleic acids, and enzymatic RNA molecules such as ribozymes. Each of these compounds can be targeted to a given miRNA and destroy or induce the destruction of the target miRNA. For example, expression of a given miRNA can be inhibited by inducing RNA interference of the miRNA with an isolated double-stranded RNA (“dsRNA”) molecule which has at least 90%, for example 95%, 98%, 99% or 100%, sequence homology with at least a portion of the miRNA. In a preferred embodiment, the dsRNA molecule is a “short or small interfering RNA” or “siRNA.”

siRNA useful in the present methods comprise short double-stranded RNA from about 17 nucleotides to about 29 nucleotides in length, preferably from about 19 to about 25 nucleotides in length. The siRNA comprise a sense RNA strand and a complementary antisense RNA strand annealed together by standard Watson-Crick base-pairing interactions (hereinafter “base-paired”). The sense strand comprises a nucleic acid sequence which is substantially identical to a nucleic acid sequence contained within the target miRNA.

As used herein, a nucleic acid sequence in a siRNA which is “substantially identical” to a target sequence contained within the target mRNA is a nucleic acid sequence that is identical to the target sequence, or that differs from the target sequence by one or two nucleotides. The sense and antisense strands of the siRNA can comprise two complementary, single-stranded RNA molecules, or can comprise a single molecule in which two complementary portions are base-paired and are covalently linked by a single-stranded “hairpin” area. The siRNA can also be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides.

One or both strands of the siRNA can also comprise a 3′ overhang. As used herein, a “3′ overhang” refers to at least one unpaired nucleotide extending from the 3′-end of a duplexed RNA strand. Thus, in one embodiment, the siRNA comprises at least one 3′ overhang of 1 to about 6 nucleotides (which includes ribonucleotides or deoxyribonucleotides) in length, preferably from 1 to about 5 nucleotides in length, more preferably from 1 to about 4 nucleotides in length, and particularly preferably from about 2 to about 4 nucleotides in length. In a preferred embodiment, the 3′ overhang is present on both strands of the siRNA, and is 2 nucleotides in length. For example, each strand of the siRNA can comprise 3′ overhangs of dithymidylic acid (“TT”) or diuridylic acid (“uu”).

The siRNA can be produced chemically or biologically, or can be expressed from a recombinant plasmid or viral vector, as described above for the isolated miRNAs. Exemplary methods for producing and testing dsRNA or siRNA molecules are described in U.S. published patent application 2002/0173478 to Gewirtz and in U.S. published patent application 2004/0018176 to Reich et al., the entire disclosures of which are herein incorporated by reference.

Expression of a given miRNA can also be inhibited by an antisense nucleic acid. As used herein, an “antisense nucleic acid” refers to a nucleic acid molecule that binds to target RNA by means of RNA-RNA or RNA-DNA or RNA-peptide nucleic acid interactions, which alters the activity of the target RNA. Antisense nucleic acids suitable for use in the present methods are single-stranded nucleic acids (e.g., RNA, DNA, RNA-DNA chimeras, PNA) that generally comprise a nucleic acid sequence complementary to a contiguous nucleic acid sequence in a miRNA. Preferably, the antisense nucleic acid comprises a nucleic acid sequence that is 50-100% complementary, more preferably 75-100% complementary, and most preferably 95-100% complementary to a contiguous nucleic acid sequence in an miRNA. Nucleic acid sequences for the miRNAs are provided in Table A. Without wishing to be bound by any theory, it is believed that the antisense nucleic acids activate RNase H or some other cellular nuclease that digests the miRNA/antisense nucleic acid duplex.

In a preferred embodiment the inhibitor is an antagomir and/or an antisense oligonucleotide.

The term “antagomir” as used herein refers to a chemically engineered small RNA that is used to silence miR-21, miR-92a-1, miR-92a-2, miR-92b, miR-126, miR-181a, miR-320c-1, miR-320c-2 and miR-1908. The antagomir is complementary to the specific miRNA target with either mis-pairing or some sort of base modification. Antagomirs may also include some sort of modification to make them more resistant to degradation. In a preferred embodiment the antagomir is a chemically engineered cholesterol-conjugated single-stranded RNA analogue.

Inhibition of miRNAs can also be achieved with antisense 2′-O-methyl (2′-O-Me) oligoribonucleotides, 2′-O-methoxyethyl (2′-O-MOE), phosphorothioates, locked nucleic acid (LNA), morpholino oligomers or by use of lentivirally or adenovirally expressed antagomirs (Stenvang and Kauppinen (2008), Expert Opin. Biol. Ther. 8(1):59-81). Furthermore, MOE (2′-O-methoxyethyl phosphorothioate) or LNA (locked nucleic acid (LNA) phosphorothioate chemistry)-modification of single-stranded RNA analogous can be used to inhibit miRNA activity.

Antisense nucleic acids can also contain modifications of the nucleic acid backbone or of the sugar and base moieties (or their equivalent) to enhance target specificity, nuclease resistance, delivery or other properties related to efficacy of the molecule. Such modifications include cholesterol moieties, duplex intercalators such as acridine or the inclusion of one or more nuclease-resistant groups.

Antisense nucleic acids can be produced chemically or biologically, or can be expressed from a recombinant plasmid or viral vector, as described above for the isolated miRNAs. Exemplary methods for producing and testing are within the skill in the art; see, e.g., Stein and Cheng (1993), Science 261:1004 and U.S. Pat. No. 5,849,902 to Woolf et al., the entire disclosures of which are herein incorporated by reference.

Expression of a given miRNA can also be inhibited by an enzymatic nucleic acid. As used herein, an “enzymatic nucleic acid” refers to a nucleic acid comprising a substrate binding region that has complementarity to a contiguous nucleic acid sequence of an miRNA, and which is able to specifically cleave the miRNA. Preferably, the enzymatic nucleic acid substrate binding region is 50-100% complementary, more preferably 75-100% complementary, and most preferably 95-100% complementary to a contiguous nucleic acid sequence in a miRNA. The enzymatic nucleic acids can also comprise modifications at the base, sugar, and/or phosphate groups. An exemplary enzymatic nucleic acid for use in the present methods is a ribozyme.

The enzymatic nucleic acids can be produced chemically or biologically, or can be expressed from a recombinant plasmid or viral vector, as described above for the isolated miRNAs. Exemplary methods for producing and testing dsRNA or siRNA molecules are described in Werner and Uhlenbeck (1995), Nucl. Acids Res. 23:2092-96; Hammann et al. (1999), Antisense and Nucleic Acid Drug Dev. 9:25-31; and U.S. Pat. No. 4,987,071 to Cech et al, the entire disclosures of which are herein incorporated by reference.

The miRNAs or miRNA expression inhibiting compounds can be administered to a patient by any means suitable for delivering these compounds to endothelial cells of the patient. For example, the miRNAs or miR expression inhibiting compounds can be administered by methods suitable to transfect cells of the patient with these compounds, or with nucleic acids comprising sequences encoding these compounds. Preferably, the cells are transfected with a plasmid or viral vector comprising sequences encoding at least one miRNA or miRNA expression inhibiting compound.

The miRNA or miRNA expression inhibiting compound can also be administered to a patient by any suitable enteral or parenteral administration route. Suitable enteral administration routes for the present methods include, e.g., oral, rectal, or intranasal delivery. Suitable parenteral administration routes include, e.g., intravascular administration (e.g., intravenous bolus injection, intravenous infusion, intra-arterial bolus injection, intra-arterial infusion and catheter instillation into the vasculature); peri- and intra-tissue injection (e.g., intra-retinal injection, or subretinal injection); subcutaneous injection or deposition, including subcutaneous infusion (such as by osmotic pumps); direct application to the tissue of interest, for example by a catheter or other placement device (e.g., a retinal pellet or a suppository or an implant comprising a porous, non-porous, or gelatinous material); and inhalation. Preferred administration routes are injection, infusion and direct injection into the cardiovascular tissue.

In the present methods, an miRNA or miRNA expression inhibiting compound can be administered to the patient either as naked RNA, in combination with a delivery reagent, or as a nucleic acid (e.g., a recombinant plasmid or viral vector) comprising sequences that express the miRNA or the miRNA expression inhibiting compound. Suitable delivery reagents include, e.g, the Minis Transit TKO lipophilic reagent; lipofectin; lipofectamine; cellfectin; polycations (e.g., polylysine), and liposomes.

Recombinant plasmids and viral vectors comprising sequences that express the miRNAs or miRNA expression inhibiting compounds, and techniques for delivering such plasmids and vectors to cardiovascular cells, are discussed above.

In a preferred embodiment, liposomes are used to deliver a miRNA or miRNA expression inhibiting compound (or nucleic acids comprising sequences encoding them) to a patient. Liposomes can also increase the blood half-life of the gene products or nucleic acids. Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream.

A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference. The liposomes for use in the present methods can comprise a ligand molecule that targets the liposome to cardiovascular cells. Ligands which bind to receptors prevalent in cardiovascular cells, such as monoclonal antibodies that bind to cardiovascular cell antigens, are preferred. The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. In a particularly preferred embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference. Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; animated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive animation using Na(CN)BH3 and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects will efficiently accumulate these liposomes; see Gabizon, et al. (1988), Proc. Natl. Acad. Sci., USA, 18:6949-53. In addition, the reduced uptake by the RES lowers the toxicity of stealth liposomes by preventing significant accumulation of the liposomes in the liver and spleen. Thus, liposomes that are modified with opsonization-inhibition moieties are particularly suited to deliver the miRNAs or miRNA expression inhibition compounds (or nucleic acids comprising sequences encoding them) to cardiovascular cells.

### Pharmaceutical Compositions

The invention relates to a pharmaceutical composition comprising a compound or a vector that raises the expression level of one miRNA for use in the treatment of atherosclerosis in a patient in need thereof wherein said miRNA is selected from the group consisting of miR-146a, miR-302c, miR-372 and miR-624. Combination of compounds or vectors that raise the expression level of said miRNAs are also encompassed by the invention for use in the treatment of atherosclerosis in a patient in need thereof.

The invention also relates to a pharmaceutical composition comprising a compound or a vector that inhibit the expression level of one miRNA for use in the treatment of atherosclerosis in a patient in need thereof wherein said miRNA is selected from the group consisting of miR-92b, miR-126, miR-181a, miR-320c-1, miR-320c-2 and miR-1908. Combination of compounds or vectors that inhibit the expression level of said miRNAs are also encompassed by the invention for use in the treatment of atherosclerosis in a patient in need thereof.

The invention also relates to a pharmaceutical composition comprising a compound or a vector that raises the expression level of one miRNA selected from the group consisting of miR-146a, miR-302c, miR-372 and miR-624 or a compound or a vector that inhibit the expression level of one miRNA selected from the group consisting of miR-92b, miR-126, miR-181a, miR-320c-1, miR-320c-2 and miR-1908 for use in the treatment of atherosclerosis in a patient in need thereof. Combination of compounds or vectors that raise or inhibit the expression level of said miRNAs are also encompassed by the invention for use in the treatment of atherosclerosis in a patient in need thereof.

In one embodiment, the pharmaceutical composition according to the invention may further comprise a compound or a vector that inhibit the expression level of miRNA for use in the treatment of atherosclerosis in a patient in need thereof wherein said miRNA is selected from the group consisting of miR-21, miR-92a-1 and miR-92a-2 or a combination thereof. Combination of compounds or vectors that inhibit the expression level of said miRNAs are also encompassed by the invention for use in the treatment of atherosclerosis in a patient in need thereof.

The miRNA of the invention (or nucleic acids or vectors according to the invention) may be administered in the form of a pharmaceutical composition, as defined below.

The miRNAs or miRNA expression inhibition compounds of the invention are preferably formulated as pharmaceutical compositions, prior to administering to a patient, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations comprise at least one miRNA or miRNA expression inhibition compound (or at least one nucleic acid comprising sequences encoding them) (e.g., 0.1 to 90% by weight), or a physiologically acceptable salt thereof, mixed with a pharmaceutically-acceptable carrier. The pharmaceutical formulations of the invention can also comprise at least one miRNA or miRNA expression inhibition compound (or at least one nucleic acid comprising sequences encoding them) which are encapsulated by liposomes and a pharmaceutically-acceptable carrier. Preferred pharmaceutically-acceptable carriers are water, buffered water, normal saline, 0.4% saline, 0.3% glycine, hyaluronic acid and the like.

In a particular embodiment, the pharmaceutical compositions of the invention comprise at least one miRNA or miRNA expression inhibition compound (or at least one nucleic acid comprising sequences encoding them) which is resistant to degradation by nucleases. One skilled in the art can readily synthesize nucleic acids which are nuclease resistant, for example by incorporating one or more ribonucleotides that are modified at the 2′-position into the miRNAs. Suitable 2′-modified ribonucleotides include those modified at the 2′-position with fluoro, amino, alkyl, alkoxy, and O-allyl.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include, e.g., physiologically biocompatible buffers (e.g., tromethamine hydrochloride), additions of chelants (such as, for example, DTPA or DTPA-bisamide) or calcium chelate complexes (such as, for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid pharmaceutical compositions of the invention, conventional nontoxic solid pharmaceutically acceptable carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25%-75%, of the at least one miRNA or miRNA expression inhibition compound (or at least one nucleic acid comprising sequences encoding them). A pharmaceutical composition for aerosol (inhalational) administration can comprise 0.01%-20% by weight, preferably 1%-10% by weight, of the at least one miRNA or miRNA expression inhibition compound (or at least one nucleic acid comprising sequences encoding them) encapsulated in a liposome as described above, and a propellant. A carrier can also be included as desired; e.g., lecithin for intranasal delivery.

### Diagnostics Methods According to the Invention

A further aspect of the invention relates to a method of identifying a patient having or at risk of having or developing atherosclerosis, comprising a step of measuring in a sample obtained from said patient the expression level of at least one miRNA selected from the groups consisting of miR-21, miR-92a-1, miR-92a-2, miR-92b, miR-126, miR-146a, miR-155, miR-181a, miR-302c, miR-320c-1, miR-320c-2, miR-372, miR-624 and miR-1908.

Typically, 1, 2, 3, 4, 5, 6, 7, 8, 9, 10, 11, 12, 13 or 14 miRNAs are measured.

A further aspect of the invention relates to a method of identifying a patient having or at risk of having or developing atherosclerosis, comprising a step of measuring in a sample obtained from said patient the expression level of all miRNAs of the group consisting of miR-21, miR-92a-1, miR-92a-2, miR-92b, miR-126, miR-146a, miR-155, miR-181a, miR-302c, miR-320c-1, miR-320c-2, miR-372, miR-624 and miR-1908.

The method of the invention may further comprise a step consisting of comparing the expression level of at least one miRNA in the sample with a control, wherein detecting differential in the expression level of the miRNA between the sample and the control is indicative of patient having or at risk of having or developing an atherosclerosis. The control may consist in sample associated with a healthy patient not afflicted with atherosclerosis or in a sample associated with a patient afflicted with atherosclerosis.

In one embodiment, high expression level of at least one miRNA selected from the group consisting of miR-146a, miR-302c, miR-372 and miR-624 and low expression level of at least one miRNA selected from the group consisting of miR-21, miR-92a-1, miR-92a-2, miR-92b, miR-126, miR-181a, miR-320c-1, miR-320c-2 and miR-1908 is indicative of patient not having or at risk of having or developing an atherosclerosis.

In another embodiment, low expression level of at least one miRNA selected from the group consisting of miR-146a, miR-302c, miR-372 and miR-624 and high expression level of at least one miRNA selected from the group consisting of miR-21, miR-92a-1, miR-92a-2, miR-92b, miR-126, miR-181a, miR-320c-1, miR-320c-2 and miR-1908 is indicative of patient having or at risk of having or developing an atherosclerosis.

According to the invention, measuring the expression level of the miRNA of the invention in the sample obtained form the patient can be performed by a variety of techniques.

For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted miRNAs is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of Amplification include ligase chain reaction (LCR), transcription-mediated amplification (TMA), strand displacement amplification (SDA) and nucleic acid sequence based amplification (NASBA).

In a particular embodiment, the determination comprises contacting the sample with selective reagents such as probes or primers and thereby detecting the presence, or measuring the amount of miRNAs originally in the sample. Contacting may be performed in any suitable device, such as a plate, microtiter dish, test tube, well, glass, column, and so forth In specific embodiments, the contacting is performed on a substrate coated with the reagent, such as a miRNA array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a miRNAs hybrid, to be formed between the reagent and the miRNAs of the sample.

Nucleic acids exhibiting sequence complementarity or homology to the miRNAs of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin).

The probes and primers are “specific” to the miRNAs they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

Accordingly, the present invention concerns the preparation and use of miRNA arrays or miRNA probe arrays, which are macroarrays or microarrays of nucleic acid molecules (probes) that are fully or nearly complementary or identical to a plurality of miRNA molecules positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of miRNA-complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass, metal, plastic, latex, and silicon. Such arrays may vary in a number of different ways, including average probe length, sequence or types of probes, nature of bond between the probe and the array surface, e.g. covalent or non-covalent, and the like.

After an array or a set of miRNA probes is prepared and/or the miRNA in the sample or miRNA probe is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001). Of particular interest in many embodiments is the use of stringent conditions during hybridization. Stringent conditions are known to those of skill in the art.

Alternatively, miRNAs quantification method may be performed by using stem-loop primers for reverse transcription (RT) followed by a real-time TaqMan® probe. Typically, said method comprises a first step wherein the stem-loop primers are annealed to miRNA targets and extended in the presence of reverse transcriptase. Then miRNA-specific forward primer, TaqMan® probe, and reverse primer are used for PCR reactions. Quantitation of miRNAs is estimated based on measured CT values.

Many miRNA quantification assays are commercially available from Qiagen (S. A. Courtaboeuf, France) or Applied Biosystems (Foster City, USA).

Expression level of a miRNA may be expressed as absolute expression level or normalized expression level. Typically, expression levels are normalized by correcting the absolute expression level of a miRNA by comparing its expression to the expression of a mRNA that is not a relevant for determining patient having or at risk of having or developing an atherosclerosis, e.g., a housekeeping mRNA that is constitutively expressed. Suitable mRNA for normalization include housekeeping mRNAs such as the U6, U24, U48 and S18. This normalization allows the comparison of the expression level in one sample, e.g., a patient sample, to another sample, or between samples from different sources.

A method of treating atherosclerosis in a patient in need thereof comprising the steps of:

i) providing a sample from a patient,

ii) measuring the expression level of at least one miRNA selected from the group consisting of miR-21, miR-92a-1, miR-92a-2, miR-92b, miR-126, miR-146a, miR-155, miR-181a, miR-302c, miR-320c-1, miR-320c-2, miR-372, miR-624 and miR-1908 in the sample obtained at step i),

iii) comparing said expression level measured in step ii) with a control, wherein low expression level of at least one miRNA selected from the group consisting of miR-146a, miR-302c, miR-372 and miR-624 and high expression level of at least one miRNA selected from the group consisting of miR-21, miR-92a-1, miR-92a-2, miR-92b, miR-126, miR-181a, miR-320c-1, miR-320c-2 and miR-1908 is indicative of patient having or at risk of having or developing an atherosclerosis, and

iv) treating said patient having or at risk of having or developing an atherosclerosis with a compound according to the invention and/or an atherosclerosis treatment.

A further aspect of the invention relates to a method for monitoring the efficacy of a treatment for a cardiovascular disease in a patient in need thereof.

Methods of the invention can be applied for monitoring the treatment (e.g., drug compounds) of the patient. For example, the effectiveness of an agent to affect the expression level of the miRNA (as herein after described) according to the invention can be monitored during treatments of patients receiving atherosclerosis treatments.

The “atherosclerosis treatment” that is referred to in the definition of step a) above relate to any type of atherosclerosis therapy undergone by the atherosclerosis patients previously to collecting the atherosclerosis tissue samples, including statins, antioxidant and surgery, e.g. angioplasty procedure.

Accordingly, the present invention relates to a method for monitoring the treatment of patient affected with an atherosclerosis, said method comprising the steps consisting of:

i) diagnosis of atherosclerosis before said treatment by performing the method of the invention

ii) diagnosis of atherosclerosis after said treatment by performing the method of the invention

iii) and comparing the results determined a step i) with the results determined at step ii) wherein a difference between said results is indicative of the effectiveness of the treatment.

### Kits

The invention also relates to kits for performing the methods of the invention, wherein said kits comprise a compound that raises the expression level of one miRNA selected from the group consisting of miR-146a, miR-302c, miR-372, miR-624 or a compound that inhibits the expression level of one miRNA selected from the group consisting of miR-92b, miR-126, miR-181a, miR-320c-1, miR-320c-2 and miR-1908 for use in the treatment of atherosclerosis in a patient in need thereof. Combination of compounds that raise the expression level of said miRNAs and compounds that inhibit the expression level of said miRNAs are also encompassed by the invention for use in the treatment of atherosclerosis in a patient in need thereof.

In one embodiment, the kit of the invention may further comprise one compound that inhibits the expression level of one miRNA selected from the group consisting of miR-21, miR-92a-1, miR-92a-2. Combination of compounds that inhibit the expression level of said miRNAs are also encompassed by the invention for use in the treatment of atherosclerosis in a patient in need thereof.

A further object of the invention relates to kits for performing the methods of the invention, wherein said kits comprise means for measuring the expression level of the miRNA clusters of the invention in the sample obtained from the patient. The kits may include probes, primers macroarrays or microarrays as above described.

For example, the kit may comprise a set of miRNA probes as above defined, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards.

Alternatively the kit of the invention may comprise amplification primers (e.g. stem-loop primers) that may be pre-labelled or may contain an affinity purification or attachment moiety. The kit may further comprise amplification reagents and also other suitably packaged reagents and materials needed for the particular amplification protocol.

In a particular embodiment, the kit of the invention relates to a kit for identifying whether a patient has or is at risk of having or developing an atherosclerosis, comprising means for measuring, in a sample obtained from said patient, at least one miRNA selected from the group consisting of miR-21, miR-92a-1, miR-92a-2, miR-92b, miR-126, miR-146a, miR-155, miR-181a, miR-302c, miR-320c-1, miR-320c-2, miR-372, miR-624 and miR-1908.

In a particular embodiment, the kit of the invention relates to a kit for identifying whether a patient has or is at risk of having or developing an atherosclerosis, comprising means for measuring, in a sample obtained from said patient, at least two miRNA selected from the group consisting of miR-21, miR-92a-1, miR-92a-2, miR-92b, miR-126, miR-146a, miR-155, miR-181a, miR-302c, miR-320c-1, miR-320c-2, miR-372, miR-624 and miR-1908.

In a particular embodiment, the kit of the invention relates to a kit which further comprise means for comparing the expression level of the miRNA in the sample with a control, wherein detecting differential in the expression level of the miRNA between the sample and the control is indicative of a risk of having or developing an atherosclerosis. The control may consist in sample associated with a healthy patient not afflicted with atherosclerosis or in a sample associated with a patient afflicted with atherosclerosis.

The invention will be further illustrated by the following examples. However, these examples should not be interpreted in any way as limiting the scope of the present invention.

### EXAMPLE

Endothelial microRNA Expression Profile as a Function of Shear Stress and oxLDL Stimulation

It is well recognized that gene expression is differentially modulated by different flow conditions at the endothelial level. To determine if microRNAs (miR), a novel class of gene regulator, could be involved in this process, we performed a microarray analysis. For this, we used human endothelial cells (HUVEC) exposed to low shear stress (LSS, 1.5 dyne/cm2) or high shear stress (HSS, 10 dynes/cm2) in a parallel plate chamber apparatus for 24 hours. To mimic pro-atherogenic conditions in vitro, we also performed stimulation with oxidized LDL (oxLDL, 25 μg/ml) in both conditions (LSS and HSS). All conditions were compared to static conditions treated or not with oxLDL.

Microarray's analysis showed that a large amount of miRs is modulated by flow conditions but also by oxLDL stimulation. In order to select most relevant miRs, we next realised a biostatistical analysis and apllied the following criterias:
1—modulation—positively or negatively—as a function of flow,
2—a differential variation under oxLDL stimulation. By applying these criteria, we determined that only a small set of miRs could be considered as interesting (Table 1).

TABLE 1
Selected microRNAs after biostatistical analysis.

| | | Δ fonction de la stimulation |
|---|---|---|
| miR | Δ fonction du flux | par les oxLDL |
| miR-21 | [symbol] | [symbol] |
| miR-302c | [symbol] | [symbol] |
| miR-320c | [symbol] | [symbol] |
| miR-372 | [symbol] | [symbol] |
| miR-624 | [symbol] | [symbol] |
| miR-1908 | [symbol] | [symbol] |

miRs have been identified by biostastical analysis and exhibit criterias mentioned above. Δ = variation observed between LSS and HSS. Arrows indicate modulation observed.

After identifying miRs of interest for this study, the inventors quantify their relative expression in all the different experimental conditions by quantitative PCR. Only those presenting a strong concordance between the variations of interactions observed by the analysis by microarray and the validation by qPCR will be considered. After this, the inventors selected the miR-21, miR-92, miR-126, miR-320, miR-155, miR-181a, miR-146a.

In order to determine the impact of a selected miRNA during atherosclerosis development, the inventors investigate the impact of an antagomiR treatment on the setting of atherosclerosis. For this, hypercholestermic mice (LDLr −/−) under high fat diet receive via intravenous injection specific antagomiR (16 mg/kg) for 10 weeks. Analysis of atherosclerotic lesions sizes and locations are performed.

### REFERENCES

Throughout this application, various references describe the state of the art to which this invention pertains. The disclosures of these references are hereby incorporated by reference into the present disclosure.

1. A method for the treatment of arthrosclerosis in a patient in need thereof comprising a step of administering to the patient
a) a compound that raises an expression level of one or more miRNAs selected from the group consisting of miR-146a, miR-302c, miR-372, and miR-624, or
b) a compound that inhibits an expression level of one or more miRNAs selected from the group consisting of miR-92b, miR-126, miR-181a, miR-320c-1, miR-320c-2 and miR-1908, or or
c) both a) and b).

2. The method according to the claim 1 further comprising a step of administering a compound that inhibits an expression level of one or more miRNAs selected from the group consisting of miR-21, miR-92a-1, miR-92a2.

3-4. (canceled)

5. A method of identifying a patient having or at risk of having or developing atherosclerosis, comprising the steps of
a) measuring, in a sample obtained from said patient, an expression level of at least one miRNA selected from the group consisting of miR-21, miR-92a-1, miR-92a-2, miR-92b, miR-126, miR-146a, miR-155, miR-181a, miR-302c, miR-320c-1, miR-320c-2, miR-372, miR-624 and miR-1908,
b) comparing the expression level of the at least one miRNA to a control miRNA expression level, and
c) detecting a differential in the expression level of the at least one miRNA and the a control miRNA expression level, wherein detection of a differential indicates that the patient has or is at risk of having or developing atherosclerosis.

6-7. (canceled)

8. The method according to claim 5 wherein the patient is afflicted with a coronary disorder, a vascular disorder, atherosclerotic vascular disease, aneurysm, stroke, an asymptomatic coronary artery disease, an chronic ischemic disorder without myocardial necrosis, stable or effort angina pectoris, an acute ischemic disorder, myocardial necrosis, unstable angina pectoris, an ischemic disorder, and myocardial infarction.

9. The method according to claim 1, wherein the compound that raises the expression level of one or more miRNAs is a vector comprising nucleic acid sequences encoding the one or more miRNAs.

10. The method according to claim 1 wherein the patient is afflicted with one or more of a coronary disorder, a vascular disorder, atherosclerotic vascular disease, aneurysm, stroke, an asymptomatic coronary artery disease, a chronic ischemic disorder without myocardial necrosis, stable or effort angina pectoris, an acute ischemic disorder, myocardial necrosis, unstable angina pectoris, an ischemic disorder and myocardial infarction.

11. The method according to claim 5, wherein expression levels of at least two miRNAs are measured.

12. A method for monitoring the efficacy of a treatment for cardiovascular disease in a patient in need thereof, comprising the steps of:
a) measuring, in a sample obtained from the patient, an expression level of at least one miRNA selected from the group consisting of miR-21, miR-92a-1, miR-92a-2, miR-92b, miR-126, miR-146a, miR-155, miR-181a, miR-302c, miR-320c-1, miR-320c-2, miR-372, miR-624 and miR-1908,
b) comparing the expression level of the at least one miRNA with a control miRNA expression level 1,
c) detecting a differential between the expression level of the at least one miRNA and the control miRNA expression level, the differential indicating that the patient has or is at risk of having or developing atherosclerosis,
d) treating the patient for atherosclerosis and then
e) measuring, in a sample obtained from said patient, an expression level of at least one miRNA selected from the group consisting of miR-21, miR-92a-1, miR-92a-2, miR-92b, miR-126, miR-146a, miR-155, miR-181a, miR-302c, miR-320c-1, miR-320c-2, miR-372, miR-624 and miR-1908,
f) comparing the expression level of the at least one expression level of miRNA measured in step e) with the at least one expression level of miRNA measured in step a), wherein detection of a differential between the expression level of the at least one measured in step e) and the at least one expression level of miRNA measured in step a) is indicative of the efficacy of the treatment.

13. The method according to claim 12, wherein expression levels of at least two miRNAs are measured in steps a) and e).
